(19) United States
(12) Patent Application Publication
(10) Pub. No.: US 2008/0097297 A1
(43) Pub. Date: Apr. 24, 2008

(54) Medical device for local site-specific drug delivery

(76) Inventors: Gregory S. Kelley, San Diego, CA (US); Show-Mean S. Wu, San Diego, CA (US)

Correspondence Address: Gregory S. Kelley, 8299 Echo Dell Road, San Diego, CA, 92119, US

(21) Appl. No.: 11/513,888

(22) Filed: Aug. 31, 2006

(51) Int. Cl. A61M 25/10; A61B 18/14; A61B 18/02; A61B 18/18

(52) U.S. Cl. 604/103.01; 606/14; 606/21; 606/27

(57) ABSTRACT

The invention discloses a medical device and methods for site-specific diagnosis, energy therapy, sampling, and drug delivery in vivo with a catheter having at least a self-expandable piercing element and a drug delivery conduit.

### FIELD OF THE INVENTION

The invention relates generally to the fields of treating the vessels or tissue of a patient. More particularly, the present invention relates to a medical device and methods for site-specific diagnosis, energy therapy, fluid sampling, and drug delivery to local target tissue.

### BACKGROUND OF THE INVENTION

The injection of medicinal agents to pre-selected sites within a patient's body is a common practice. For example, a syringe is often used in combination with a hypodermic needle to administer medicinal agent(s). The piercing tip of such a traditional needle has been carefully shaped and sharpened, both to minimize trauma as it pierces the patient's tissue and to minimize the likelihood that tissue might enter and obstruct the lumen, which serves as the conduit to allow delivery of the bioactive agent(s). The shape of the needles' piercing tip is generally constructed with an elongated bevel or bevels such that the distal-most end of the needle terminates in a sharp point formed in part by the bevel.

Previously, the Infiltrator™ angioplasty balloon catheter was introduced for treating a vessel of a patient's cardiovascular system by injecting a fluid directly into the vessel wall (LDDC-07, 21 Jul. 2004, pages 59-62). The Infiltrator balloon catheter consists of a noncompliant positioning balloon and an independently operated infiltration system that is built on the balloon surface with low crossing profile. Upon the inflation of the balloon, the miniature injection nipples penetrate the media of the vessel and predetermined doses of fluid are slowly infiltrated directly into the vessel wall with hand injection.

Prior art devices for use in conjunction with an angioplasty procedure to obviate a restenosis generally consist of an expanding member and penetrating dispensers on the expanding member. For example, one such device utilizes a balloon to position a plurality of apertures against the vessel wall near a stenosis and a medicament is released from the aperture as taught in U.S. Pat. Nos. 5,112,305, 5,681,281, 5,713,863, 5,746,716, 5,873,852, 6,102,904, 6,210,392, and 6,695,830.

U.S. Pat. No. 5,499,971 to Shapland et al., entire contents of which are incorporated herein by reference, discloses a drug delivery apparatus and method for iontophoretically delivering a drug locally to internal body tissue, including a current source producing a net flow of current in a desired direction with high frequency waveforms which enhance delivery and minimize side effects typically associated with iontophoresis.

Transcatheter drug delivery with optimal doses to a specific site in a body passageway or within body tissue enables site-specific disease treatment, such as treating benign prostate hyperplasia, post-angioplasty coronary restenosis, cancers/tumors, or electrophysiology ablation in a pulmonary vein.

U.S. Pat. No. 6,695,830 to Vigil et al., entire contents of which are incorporated herein by reference, discloses a catheter with an expanding balloon member advanced along a catheter shaft within the lumen of a body vessel until the expanding member is located adjacent to the prescribed treatment area. A plurality of dispensers is mounted on the expanding member and an extracorporeal mechanism for pumping a medicinal fluid to the dispensers through a lumen in the catheter is provided. Tissue treatment is accomplished by releasing a medicament at several predetermined locations within the vessel wall to circumferentially disperse the medicament in the vessel wall.

In light of the above, it is an object of the present invention to provide a medical device and methods for diagnosing or treating a disease site-specifically with a piercing member that has a collapsed conduit lumen for preventing tissue from obstructing the conduit and preventing contact between the medicinal agent and the tissue penetrated during transit of the device through body tissue.

### SUMMARY OF THE INVENTION

One object of the invention is to provide a medical device for delivering medicinal agent and/or receiving sampling fluids site-specifically in a body passageway or within body tissue locally. The device may further serve as a conduit for delivering therapeutic energy to a target tissue and/or receiving environmental data of the adjacent target tissue. The medicinal agent of the present invention may be in the form of liquid, gas, solids, suspensions, colloids, micro-spheres, nano-spheres, or combinations thereof. Further, the medicinal agent may broadly cover other agents, such as stem cells, drugs, progenitor cells, growth factors, proteins, peptides, enzymes, and the like.

One object of the invention is to provide a medical device that does not utilize a balloon to deploy the injectors, such as the Infiltrator™, which consists of an infiltration system built on the balloon surface, does.

Some aspects of the invention provide a medical device with site-specific features comprising at least one self-expandable piercing element sized and configured for a site-specific activity in vivo selected from the group consisting of diagnosis, sampling, energy therapy, and drug delivery, wherein the piercing element has a pre-shaped curvature that penetrates a target human tissue locally when the piercing element is deployed from a lumen of the device. In one embodiment, the energy therapy is selected from the group consisting of laser beam, heated medium, and cold medium to blast nerve tissues and interrupt the pathway of electrical signals that cause an atrial fibrillation in a pulmonary vein. In a preferred embodiment, the piercing element is adapted to deliver medicine, hydrogel, solidifiable polymer, or bio-adhesive to fill an arterial aneurysm to prevent further enlargement of a diseased area of the aneurysm. In still another embodiment, the piercing element is adapted to deliver medicine or energy to treat a lower urinary tract for firming up tissue of the treated lower urinary tract and increasing resistance to involuntary leakage.

Some aspects of the invention provide a medical device with site-specific features comprising at least one self-expandable piercing element that is sized and configured for a site-specific activity selected from the group consisting of diagnosis, sampling, energy therapy, and drug delivery, wherein the piercing element comprises a piezoelectric material on a side of the piercing element so to bend the element when an electromagnetic force is applied to the piezoelectric material.

Some aspects of the invention provide a local drug delivery device with site-specific features comprising self-expandable and retractable injectors that can do multiple injections in human arteries or an organ, wherein at least one self-expandable injector is sized and configured to provide optimal site-specific dose(s) of medication, wherein the injector has a pre-shaped curvature that penetrates a target tissue of the human arteries or the organ when the injector is deployed from a lumen of the medical device.

Some aspects of the invention provide a local drug delivery device with at least one injector having a shape memory characteristic that penetrates the target tissue when the injector is deployed. In one embodiment, the injector has a stopper near a tip of the injector and around a part of an elongate body of the injector to control a penetration depth of the injector into the tissue of the human arteries or the organ. In another embodiment, the stopper serves as a marker, wherein at least a portion of the stopper comprises a radiopaque material selected from the group consisting of gold, platinum, rhenium, iridium, rhodium, tantalum, and tungsten.

Some aspects of the invention provide a drug delivery device with at least one piercing injector having a collapsed lumen to prevent contact between the medicinal agent and interposing tissue and prevent obstruction of the delivery pathway, as the injector penetrates through tissue to the treatment site. After transit to the treatment site, the lumen is opened by application of the injection pressure.

One object of the invention is to provide a drug delivery device comprising multiple pre-shaped, self-expandable anchoring wires or springs configured for the device to position securely at about the target tissue of the arteries or the organs. In one embodiment, the drug delivery device further comprises at least one inflatable site-positioning balloon configured for the device to position securely at about the target tissue of the arteries or the organs. In another embodiment, the drug delivery device further comprises a multiple lumen shaft structure that has an outer sheath, a guidewire lumen, and at least one drug delivery lumen connected to a drug delivery pathway of the injector. In still another embodiment, the shaft of the drug delivery device is constructed and selected from the group consisting of a single layer tube made of plastic or metal material, a multilayer braided or wound tube, a spiral tube, multiple pieces of single lumen tube melted together, and a multiple lumen tube.

Some aspects of the invention provide a local drug delivery device with an injector for delivering dose(s) of medication to the target tissue, wherein the dose is formulated to shrink areas of the prostate for treating benign prostate hyperplasia (BPH) or to treat a cancerous tumor, is formulated to reduce an arterial stenosis in lieu of or after stenting, balloon angioplasty or a PTCA procedure, is formulated to reverse a buildup of plaque on walls of the coronary or peripheral artery. In one embodiment, the medication is stem cells or a growth factor adapted to be delivered to a damaged heart muscle area to heal or revive tissue functionality.

For purposes of summarizing the invention, certain aspects, advantages and novel features of the invention have been described herein above. Of course, it is to be understood that not necessarily all such advantages may be achieved in accordance with any particular embodiment of the invention. Thus, the invention may be embodied or carried out in a manner that achieves or optimizes one advantage or group of advantages as conceived or suggested herein without necessarily achieving other advantages as may be conceived or suggested herein.

All of these embodiments are intended to be within the scope of the invention herein disclosed. These and other embodiments of the invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 shows one embodiment of a medical device with multiple end piercing elements.

FIG. 2 shows a cross-sectional view, section 1-1, of the medical device of FIG. 1.

FIG. 3 shows one embodiment of a medical device with multiple end piercing elements and an anchoring finger.

FIG. 4 shows one embodiment of a medical device with multiple side piercing elements.

FIG. 5 shows one embodiment of a medical device with multiple piercing elements and a positioning balloon.

FIG. 6 shows a detailed illustration of the balloon portion and the delivery body of the medical device.

FIG. 7 shows one embodiment of a self-expandable medical device with at least two expandable arms having multiple piercing elements.

FIG. 8 shows an alternate embodiment of a self-expandable medical device with at least two expandable arms having multiple piercing elements.

FIG. 9 shows an injector of the present invention with a collapsible cap at its unfolded stage.

FIG. 10 shows an injector of the present invention with a collapsible cap at its folded stage.

### DETAILED DESCRIPTION OF EXEMPLARY EMBODIMENTS

The preferred embodiments of the invention described herein relate particularly to a medical device with multiple piercing elements with hollow passageways suitable for diagnosis, energy therapy, sampling and medicinal delivery with respect to target tissue of a patient. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described herein.

One aspect of the invention provides a probe device with an outer sheath and multiple piercing elements deployable from the sheath, each piercing element comprises a piercing tip, a shape memory delivery member having a hollow delivery channel and a proximal portion attached to a delivery body inside the outer sheath. In one embodiment, the shape memory delivery member is sized and configured to self expand radially outwardly when the piercing element is deployed out of the sheath. In another embodiment, the piercing tip and a portion of the hollow delivery channel is collapsible to prevent occlusion of the channel as the piercing element penetrates to the treatment site.

For diagnosis purposes, the piercing tip may comprise a temperature sensor, a pressure sensor, or a biochemical sensor for sensing pH values, solute concentration, electrolyte type, and the like. For therapy purposes, the piercing tip may comprise an energy delivery function, such as for delivering radiofrequency energy, ultrasonic energy, microwave energy, laser energy, cryogenic energy or combinations thereof through electric wires, transducers, or optic fibers. For sampling purposes, the piercing tip may serve to intake surrounding fluid to an external assay instrument or to analyze surrounding fluid in situ, such as in an antibody-antigen conjugate assay process. For medicinal delivery, the piercing tip serves as an injector to load or deliver desired bioactive agent(s) to the surrounding tissue site-specifically and locally in vivo. The bioactive agent may further include micro-beads or nano-beads of radioactive material for irradiation therapy. Delivery of medicinal agent may be accomplished by an extracorporeal pumping operation.

The term “piercing” is broadly defined herein as an activity used in penetrating into a tissue for intended functions, including diagnosis, therapy, sampling and medicinal delivery with respect to a target tissue locally inside the body of a patient.

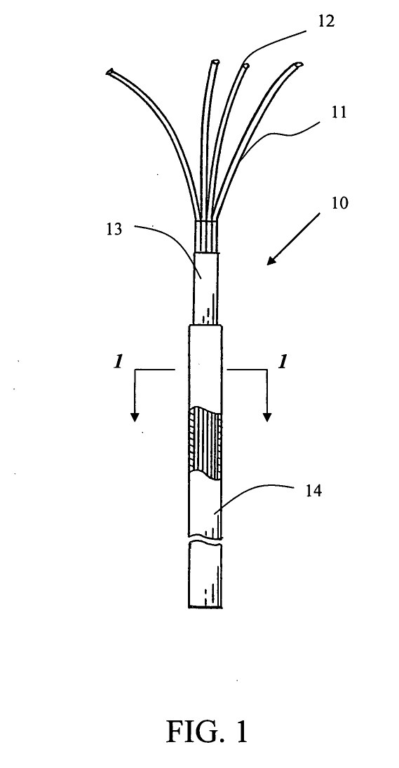

FIG. 1 shows one embodiment of a medical device 10 with one or more piercing elements 11 deployable from a distal end of a sheath 14, each piercing element having a piercing tip 12 sized and configured for serving the intended purposes of diagnosis, energy therapy, sampling, medicinal delivery, and the like. In one preferred embodiment, the medical device 10 is an injector for medicinal delivery comprising a delivery body 13 located inside the catheter sheath 14. The injector elements 11 are deployed out of the sheath 14 by sheath retraction toward an operator and recovered back into the sheath by sheath advancement. In one embodiment, the piercing element is self-expandable radially outwardly out of the sheath and retractable within the sheath. In another embodiment, the piercing element has a shape memory characteristic that penetrates the target area when the piercing element is deployed.

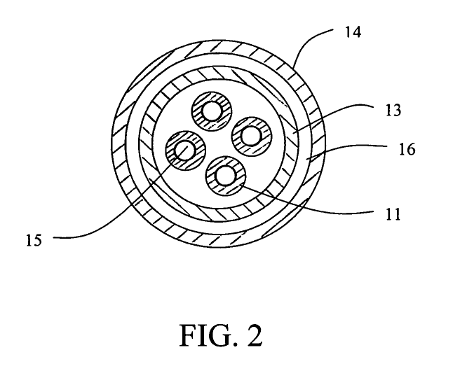

FIG. 2 shows a cross-sectional view, section 1-1, of the medical device 10 of FIG. 1. The fluid/drug delivery channel 15 of the piercing element 11 may extend to the tip 12 at the end of the medical device 10 or at the side of the medical device 30. In one embodiment, delivery channels are connected to a common lumen passageway that is in fluid communication with an external fluid source. In another embodiment, each delivery channel is in fluid communication to a separate lumen, preventing cross-flow of fluid between the lumens, which are in fluid communication with an external fluid source. The medicinal agent is injected into the target tissue by hand injection, computer programmed injection or pumping. In a preferred embodiment, there is an appropriate clearance 16 between an inner delivery body 13 and the outer sheath 14 for sheath movement.

The piercing tip 12 may be sized and configured to penetrate into the target tissue with ease and provide intended functions of diagnosis, therapy, sampling and medicinal delivery to a patient. The piercing tip of the present invention may be selected from various configurations or configured similar to a dispenser as illustrated in U.S. Pat. No. 6,695,830, entire contents of which are incorporated herein by reference. The dispenser may include a base and a penetrating section. Preferably, the dispenser is made of a metal, a ceramic, Nitinol or any sturdy material. In one embodiment, the penetrating section is defined by an opening which is opposite the base. In another embodiment, the penetrating section of the dispenser is substantially annular shaped or substantially conical shaped. In an alternative embodiment, the penetrating section is defined by an opening which extends through the side(s) of the piercing tip. In a further embodiment, the piercing tip is made of a porous material. Thus, the porous material defines the penetrating section of the dispenser, wherein the fluid medicament is forced through the pores of the porous tip section.

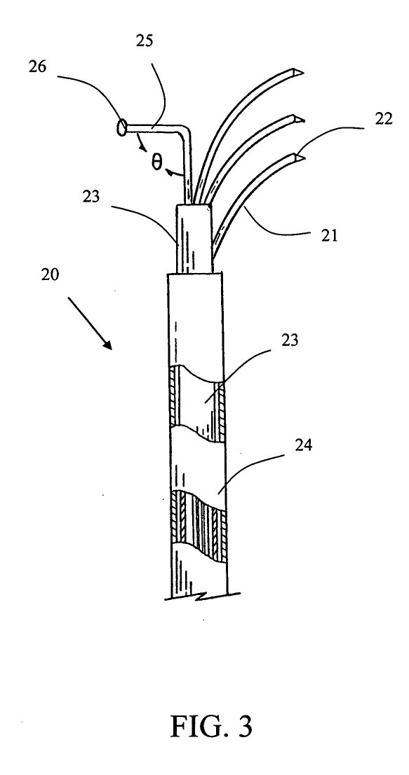

FIG. 3 shows one embodiment of a medical device 20 having a catheter sheath 24 and multiple end piercing elements 21 within the sheath, each element having a piercing tip 22, an anchoring finger 25 (also known as a pre-shaped, self-expandable anchoring wire or spring) having an anchoring base 26 or blunt tip to position against a target tissue, and a delivery body that secures the proximal ends of the piercing elements 21. The delivery body 23 is located inside the catheter sheath 24. The piercing elements 21 and the anchoring finger 25 are deployed out of the sheath 14 by the process of sheath retraction and retracted within the sheath by sheath advancement. The anchoring finger 25 may be configured outwardly curved opposite to the general orientation or direction of the piercing element to stabilize the piercing tip against the target tissue of the patient. In one embodiment, the anchoring finger 25 may comprise an angle, θ, with a thinner cross section diameter at the angle. The angle θ may be between about 91 degrees to 179 degrees. The exterior surface of the anchoring base 26 may be studded or roughened to enhance positioning ability.

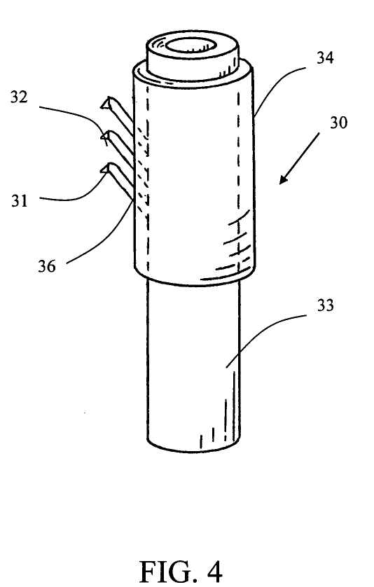

FIG. 4 shows one embodiment of a medical device 30 with multiple side piercing elements 31 attached to an inner delivery body 33 that is located within a catheter sheath 34. The piercing elements are substantially parallel to the inner delivery body adjacent the opening 36 where the elements exit and curve outwardly from the opening of the sheath. Each piercing element comprises a piercing tip 32. In operations, the piercing elements 31 along with the delivery body 33 are retracted and maintained within the catheter sheath 34 during a delivery phase to the target site. Once at the target site, the sheath 34 is retracted to deploy the piercing element(s) and the associated piercing tip for intended medical purposes. During the device withdrawal phase, the piercing elements along with the delivery body are retracted and maintained within the catheter sheath.

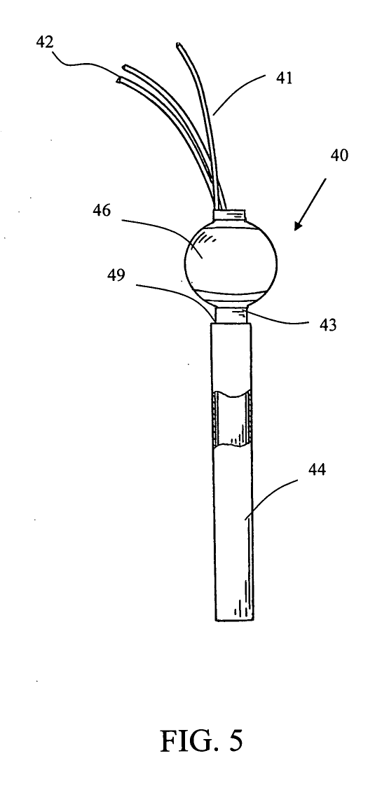

FIG. 5 shows one embodiment of a medical device 40 with multiple piercing elements 41 and an anchoring balloon 46 secured to a delivery body 43 that is located within a catheter sheath 44. In operations, the delivery body with a deflated balloon is retracted within the catheter sheath during the device delivery phase to a target tissue site. Once at the target site, the deflated balloon and the associated piercing elements are deployed out of the sheath end 49 by retracting the sheath toward the operator. The balloon is thereafter inflated to position or anchor against a part of the surrounding tissue. When the device is delivered and used in a fluid flowing system, such as an artery or a vein, the balloon may comprise multiple external fluid passageways axially so to allow adequate continued blood flow in the artery or vein during the deployment of the device.

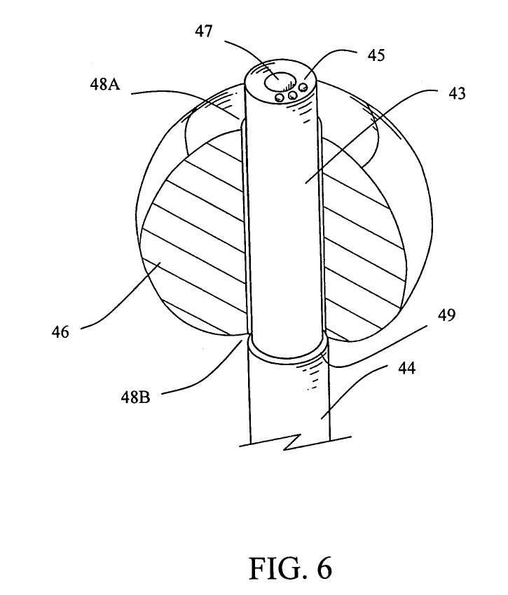

FIG. 6 shows detailed illustration of the balloon portion 46 and the delivery body 43 of the medical device 40. In one embodiment, a distal end 48A and a proximal end 48B of the balloon is securely attached and sealed to the delivery body 43. In one embodiment, each piercing element 41 comprises a lumen 45 for providing bioactive agents in the medicinal delivery mode. In another embodiment, the lumen serves as a conduit for passing the wires/optic fibers in the therapy mode or the like. In a further embodiment, the lumen serves to extract and transport fluid in the sampling mode. In still another embodiment, the lumen serves as a conduit for passing temperature measuring sensors, biochemical sensors, or the like in the diagnosis mode. The delivery body 43 may also comprise a hollow central lumen 47 for passing a guidewire or other instrument.

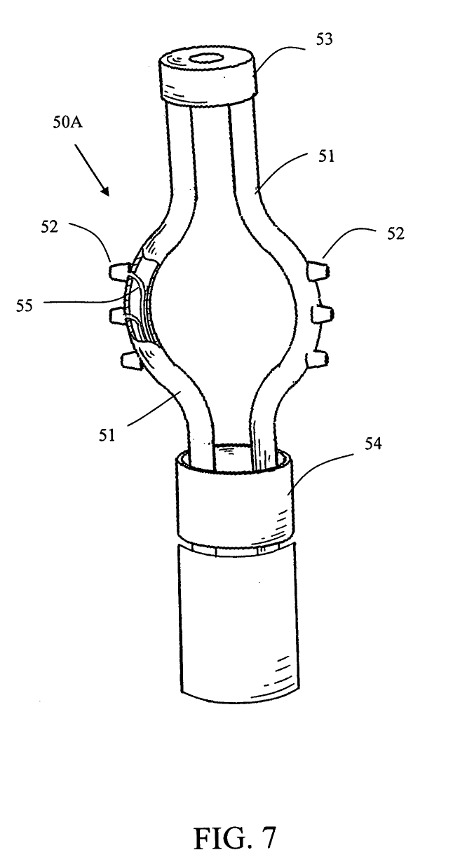

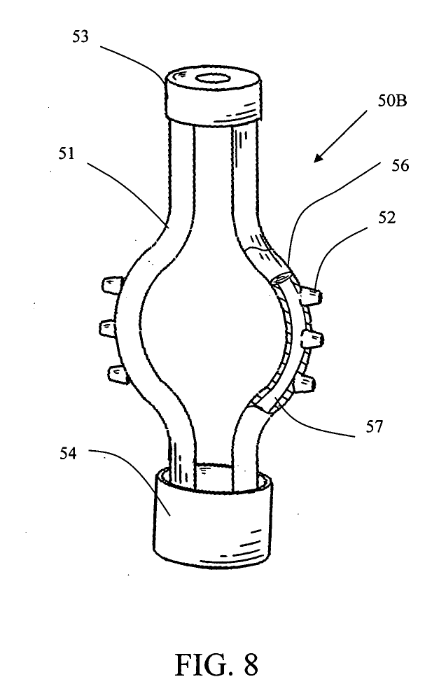

One aspect of the invention provides an injector device similar to a self-expandable stent or a multiple-arm probe. FIG. 7 shows one embodiment of a self-expandable medical device 50A with at least two expandable arms 51 having multiple piercing elements 52, whereas FIG. 8 shows an alternate embodiment of a self-expandable medical device 50B with at least two radially expandable arms having multiple piercing elements or drug delivery piercing tips. In one embodiment, the distal ends of the at least two arms are secured together at a joint 53.

Some aspects of the invention relate to a medical device with at least two expandable arms that self-expand radially outwardly when deployed, wherein at least one arm is equipped with probe/piercing elements and at least one arm has no piercing elements. In one embodiment, the at least one arm without piercing elements serves as the stabilizing or anchoring means for stabilizing and anchoring the medical device during the medical operations. In another embodiment, at least a portion of the exterior surface of the stabilizing arm comprises studded surface, textured surface, protrusion or dummy piercing tip without drug delivery capability.

As illustrated in FIG. 7, the injector device 50A comprises a hollow fluid delivery passageway 55 for each piercing element 52. The device is sized, configured and shaped to fit various anatomical conduits, such as an arterial, prostate, atrial, venous, and the like. In an alternate embodiment, as illustrated in FIG. 8, a common fluid passageway 57 is provided to all piercing elements in the same arm 51. The common fluid passageway 57 is terminated at a location 56 distal to the distal-most piercing element on the expandable arm. A catheter sheath 54 is configured to accept outwardly expandable arms 51 and their associated piercing elements 52 during a device delivery stage or retrieval stage. The delivery arm may be made of shape memory material. The shape memory arm or element has a first shape or configuration at body temperature. The shape memory arm/element is mechanically forced into a second shape when it is retracted within a catheter sheath, and the shape memory arm/element reverts to the first shape after removing the constraint, when the shape memory arm/element is deployed out of the sheath. The shape memory material and its characteristics are well known to one ordinary skilled in the art.

In one embodiment, a restrictor is placed within each piercing element 11, 21, 31, 41, or hollow fluid delivery passageway 55 or common passageway 57 to provide controlled fluid flow. By way of illustration, the restrictor may be a porous material that needs a predetermined pressure of the delivered fluid to open. Some aspects of the invention provide a medical device or an injector of the drug delivery device, wherein a restrictor or stopper 67 is placed on the piercing elements near a piercing tip to control the penetration depth of the piercing element into tissue of the human arteries or an internal organ. In one embodiment, the stopper is made from or comprises a radiopaque material selected from the group consisting of gold, platinum, rhenium, iridium, rhodium, tantalum, and tungsten. In still another embodiment, the stopper is made from a plastic filled with a radiopaque material, wherein the radiopaque material is selected from the group consisting of gold, platinum, rhenium, iridium, rhodium, tantalum, and tungsten.

The conduit of a portion of the hollow fluid delivery passageway 15, 45, 55 or the conduit of a portion of the piercing element 11, 21, 31, 41 with a hollow fluid delivery passageway may be collapsible and expandable. By ways of illustration, the conduit may be constructed of a material that is relatively flexible such as a polymer membrane, rubber, silicone or latex, which expands during delivery of the medicinal agent(s) through the conduit. After delivery of the medicinal agent(s), the conduit returns to the collapsed state. Alternatively, the collapsed conduit may be constructed with a material that is folded in the collapsed state and unfolds to allow delivery of the medicinal agent(s) through the conduit. After delivery of the medicinal agent, the conduit may return to a folded state.

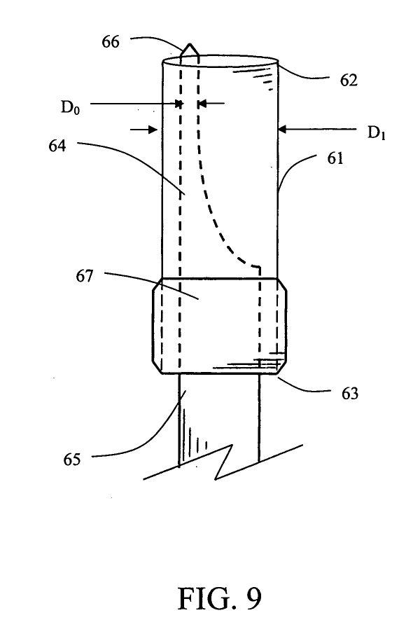

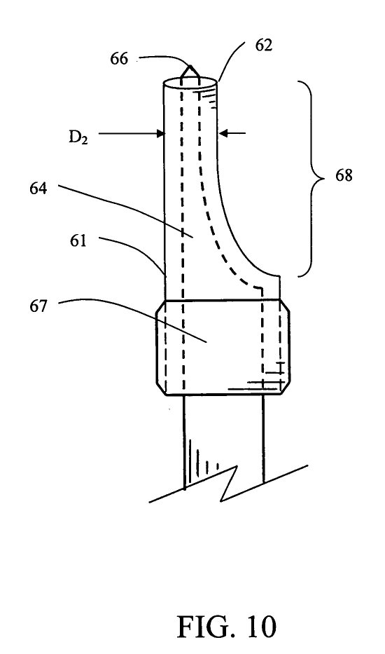

FIG. 9 shows a collapsible cap 61 at its unfolded stage that is placed on an injector 64, whereas FIG. 10 shows a collapsible cap at its folded stage on an injector to simulate the collapsed conduit discussed above. In one embodiment, the collapsible cap comprises a distal end 62, a proximal end 63, and a collapsible section 68. The collapsible cap may be made of plastic, an elastomer, or other suitable material. The proximal portion 65 of the injector may be connected to a fluid lumen that is in fluid communication with an external fluid source. The tip 66 of the injector may be pointed or sharpened for piercing purposes, wherein the tip may be sized and configured to extend an appropriate distance beyond the distal end 62 of the collapsible cap.

In one embodiment, a stopper 67 is incorporated at about the distal section of the injector 64 to control penetration depth. In operations during a device delivery stage, the collapsible cap 61 may be folded or collapsed to a diameter D₂, to prevent tissue from entering the injector 64. During delivery of medicinal agent(s) the cap 61 is unfolds or expands to a diameter D₁ which is larger than the injector diameter D₀, allowing fluid or drug to be injected to the target tissue site specifically in vivo.

Some aspects of the invention prevent tissue from obstructing the conduit during transit of the device through body tissue to the target delivery site since the conduit is collapsed during transit. Similarly, the device prevents contact between the medicinal agent and the tissue encountered during transit since the conduit is collapsed during transit toward target tissue and when withdrawn. In one embodiment, the device allows for delivery of medicinal agent(s) nearly coincident with the distal tip of the piercing element. In one embodiment, the expanded conduit conforms to anatomical restrictions, for example, when inserted through a narrow opening between relatively hard unyielding body structures. The medicinal agent(s) of the present invention may be in the form of liquid, gas, solids, suspensions, colloids, micro-spheres, nano-spheres, gels, or combinations thereof.

The major part of the piercing element is constructed with a suitably stiff material to enable it to pierce through body tissue. In one preferred embodiment, the piercing element could be constructed of a superelastic material such as nickel titanium, enabling it to undergo significant bending without permanent deformation. In another preferred embodiment, all or part of the piercing element may be constructed of a radiopaque material to facilitate imaging with fluoroscopy. It is anticipated that in most cases, the piercing element will be constructed of a metal but it could be constructed with any suitably rigid material.

Some aspects of the invention relate to methods of positioning the medical device or injector within various anatomies in a human body by inflating a side balloon or deploying a positioning member with shape memory features, wherein the positioning member is self-expandable radially outwardly when deployed from a constraint, such as a catheter sheath.

One example to make a collapsible (elastic or folded) injector is to take a pre-shaped shape memory needle and grind a distal segment axially of a length suitable for penetration, perhaps ⅔rd of the body diameter thus removed. An elastic or folded sleeve is then attached over the ground segment. This device is less occlusive within a vessel, with less blood flow impedance, versus a balloon device such as the Infiltrator. Thus, the device could be deployed for a longer time given a time sensitive procedure, for example in a coronary artery or in the atrium, than a balloon type device.

A catheter with the ability to deliver medicinal agents through a piercing tip to areas of a patient's body locally has advantages over a needle. For example, to deliver medicinal agents to the wall of a blood vessel or a small tumor, which are not readily accessible by a needle. The target area to which an agent is to be delivered may be quite localized and may be restricted. In the example of a blood vessel, it may be desirable to deliver the medicinal agent into the vessel wall at a depth of approximately 0.25 millimeters while limiting the depth of penetration to about this delivery depth to avoid perforation of the vessel wall. A traditional hypodermic needle tip does not meet this need.

In some devices disclosed herein for use in the present method, an open edge defines the penetrating section of the dispenser. In alternative devices useful for the present method and disclosed herein, each dispenser can include a porous section or an opening through the dispenser wall of the penetrating section.

Some aspects of the invention relate to a steerable needle to provide access through non-linear pathways, for example to bypass an obstruction. A steerable needle may be manufactured by incorporating a piezoelectric material on a side of the needle so to flex or bend the needle when an electromagnetic force is applied to the piezoelectric material. Some aspects of the invention provide a medical device with site-specific features comprising at least one self-expandable piercing element that is sized and configured for a site-specific activity selected from the group consisting of diagnosis, sampling, energy therapy, and drug delivery, wherein the piercing element comprises a piezoelectric material on a side of the piercing element so to flex or bend the element when an electromagnetic force is applied to the piezoelectric material.

Therapy on Benign Prostate Hyperplasia

The prostate is not round but more a dual-lobed organ with the urethra situated off center. In operations for local drug delivery in vivo, it is desirable to have a medical device with two or more elongate piercing elements positioned axially parallel to each other, wherein the two or more piercing elements protrude at approximately 120 degrees in a plane perpendicular to an axis of the device with a third positioning arm spaced at 120 degrees to the two piercing elements. The current practice enables an operator to visualize and orientate before deploying the device. The side injector (plus anchoring finger) design provides a site-specific device.

In some disease stages, such as in the prostate where deeper penetration is needed, the present invention provides easily adjustable injector length without adding crossing profile, whereas a conventional balloon based device is handicapped due to balloon size limitation. Similarly in diseases relating to an atrial fibrillation (AF) in pulmonary veins, the present invention provides easily adjustable injector length without adding crossing profile, whereas a conventional balloon catheter design is handicapped due to balloon size limitation. Other disease areas, such as a calcified peripheral or bifurcated artery are also not easily reachable by a conventional balloon catheter.

From the foregoing description, it will be appreciated that a medical device and methods for site-specific diagnosis, energy therapy, sampling, and drug delivery in vivo have been disclosed. While the components, techniques and aspects of the invention have been described with a certain degree of particularity, it is manifest that many changes may be made in the specific designs, constructions and methodology herein above described without departing from the spirit and scope of this invention.

Various modifications and applications of the invention may occur to those who are skilled in the art, without departing from the true spirit or scope of the invention. It should be understood that the invention is not limited to the embodiments set forth herein for purposes of exemplification, but is to be defined only by a fair reading of the appended claims, including the full range of equivalency to which each element thereof is entitled.

What is claimed is:

1. A medical device with site-specific features comprising at least one self-expandable piercing element sized and configured for a site-specific activity in vivo selected from the group consisting of diagnosis, sampling, energy therapy, and drug delivery.

2. The medical device of claim 1, wherein said piercing element has a pre-shaped curvature configured to penetrate a target human tissue inside a patient when said piercing element is deployed from a lumen of said device.

3. The medical device of claim 2, wherein said piercing element further comprises a piezoelectric material on a side of the piercing element so to flex or bend said element when an electromagnetic force is applied to said piezoelectric material.

4. The medical device of claim 1, wherein said energy therapy is selected from the group consisting of laser beam, heated medium, and cold medium to interrupt the pathway of electrical signals that cause an atrial fibrillation in a pulmonary vein.

5. The medical device of claim 1, wherein said piercing element is adapted to deliver medicine, hydrogel, or bio-adhesive to fill an arterial aneurysm in a patient to prevent further enlargement of a diseased area of the arterial aneurysm.

6. The medical device of claim 1, wherein said piercing element is adapted to deliver medicine or energy to treat a lower urinary tract to increase resistance to involuntary leakage.

7. A local drug delivery device with site-specific features comprising self-expandable and retractable injectors that can do multiple injections in human arteries or an internal organ, wherein at least one self-expandable injector is sized and configured to provide an optimal site-specific dose of medication.

8. The drug delivery device of claim 7, wherein said injector has a pre-shaped curvature configured to penetrate a target tissue of the human arteries or the internal organ when said injector is deployed from a lumen of said device.

9. The drug delivery device of claim 8, wherein said injector is at least in part comprised of a shape memory material.

10. The drug delivery device of claim 8, wherein said injector has a collapsed lumen that prevents occlusion of the drug delivery pathway as said injector penetrates to the target tissue and when withdrawing the device from the target tissue.

11. The drug delivery device of claim 8, wherein said injector has a stopper near a tip of the injector and around a part of an elongate body of the injector to control a penetration depth of the injector into said tissue of the human arteries or the internal organ.

12. The drug delivery device of claim 11, wherein at least a portion of the stopper is comprised of a radiopaque material selected from the group consisting of gold, platinum, rhenium, iridium, rhodium, tantalum, and tungsten.

13. The drug delivery device of claim 8, wherein said device further comprises at least one pre-shaped, self-expandable anchoring wire or spring configured to position the device securely at about the target tissue of the human arteries or the internal organs.

14. The drug delivery device of claim 8, wherein said device further comprises at least one inflatable site-positioning balloon configured to position the device securely at about the target tissue of the human arteries or the internal organs.

15. The drug delivery device of claim 8, wherein said device further comprises a multiple lumen shaft structure that has an outer sheath, a guidewire lumen, and at least one drug delivery lumen connected to a drug delivery pathway of said injector.

16. The drug delivery device of claim 15, wherein the shaft is constructed from the group consisting of single layer tube made of plastic or metal material, multilayer braided or wound tube, spiral tube, multiple pieces of tube melted together, multiple lumen tube, and combinations thereof.

17. The drug delivery device of claim 8, wherein said dose of medication is formulated to shrink an area of the prostate for treating benign prostate hyperplasia (BPH) or to treat a cancerous tumor.

18. The drug delivery device of claim 8, wherein said dose of medication is formulated to reduce an arterial stenosis in lieu of or after stenting, balloon angioplasty, or a PTCA procedure.

19. The drug delivery device of claim 8, wherein said dose of medication is formulated to reverse a buildup of plaque on walls of a coronary or peripheral artery.

20. The drug delivery device of claim 8, wherein said medication is stem cells or a growth factor adapted to be delivered to a damaged heart muscle area to heal or revive tissue functionality.
